(19) United States
(12) Patent Application Publication
(10) Pub. No.: US 2005/0089938 A1
(43) Pub. Date: Apr. 28, 2005

(54) Use of peptide fragments of the calcium channel a-1 subunit, optionally comprising mutations, for screening molecules of therapeutic interest

(76) Inventors: Jean-Marc Sabatier, Rousset (FR); Michel De Waard, Rognes (FR)

Correspondence Address: YOUNG & THOMPSON, 745 SOUTH 23RD STREET, 2ND FLOOR, ARLINGTON, VA, 22202, US

(21) Appl. No.: 10/484,885

(22) PCT Filed: Jul. 26, 2002
(86) PCT No.: PCT/FR02/02679

(30) Foreign Application Priority Data
Jul. 27, 2001 (FR) 01/10117

(51) Int. Cl. G01N 33/53; G01N 33/567; C07K 14/705

(52) U.S. Cl. 435/7.2; 530/350

(57) ABSTRACT

A subject of the present invention is the use of peptide fragments of the α-1 sub-unit of the calcium channels of mammals, of sequences derived by mutation of said fragments, or also of cells transformed by sequences coding for said fragment or derived sequences, for screening of molecules of therapeutic interest.

A subject of the present invention is the use of peptide fragments of the α-1 sub-unit of the calcium channels of mammals, sequences derived by mutation of said fragments, or also of cells transformed by sequences coding for said fragment or derived sequences, for screening molecules of therapeutic interest.

The importance of the I-II loop in the regulation of the activity of the channel has been known since 1994, the date when the anchorage site of the auxiliary β sub-unit was identified (Pragnell M. et al., (1994), “Calcium channel beta-subunit binds to a conserved motif in the I-II cytoplasmic linker of the alpha 1-subunit” Nature 368, 67-70). This β sub-unit produces an impressive number of functional modifications of the calcium channels.

The I-II cytoplasmic loop of the α-1 sub-unit of the high-threshold calcium channels which are voltage-dependent, links between them the first and the second of the four hydrophobic domains. It plays an essential role in 1) the regulation of the activity of the channel (activation and inactivation properties), 2) the level of membrane expression of the channel (control of the level of retention within the endoplasmic reticulum), 3) the regulation by the exogenous proteins (binding site of the Gβγ complex of the G proteins) and 4) the regulation by the β sub-units of the calcium channels (anchorage site of the β sub-unit and first regulation site). According to the experimental results obtained by the Inventors, it appears particularly useful to use the I-II loop of the calcium channels in order to understand the cellular roles of these calcium channels, for neuronal inactivation, or also as a pharmacological target for modulation of the activity of the calcium channels.

In fact, the invention results from the demonstration by the Inventors of the fact that the exogenous expression of the I-II loop, when it is coupled to a transmembrane segment, substantially inhibits the membrane expression of native calcium channels. This result makes it possible to reduce the expression of calcium channels in a given tissue in order to arrest the cell function of these channels.

The Inventors have demonstrated that the I-II loop is an organizational molecular loop. It is coupled to other cytoplasmic loops of the calcium channel (amino and carboxy-terminal sequences, II-III and III-IV loops). These intra-molecular interactions have the function of controlling inactivation. For example, the interaction between the I-II loop and III-IV loop of the P/Q type calcium channel plays a vital role in controlling the inactivation kinetics of the channel. In spectacular fashion, these interactions are partially or totally interrupted by the interaction of the I-II loop with the β sub-unit. In other words, the β sub-unit of the calcium channels enters into competition with the intramolecular interactions of the I-II loop. With such results, it can be expected that the Gβγ complex, which also binds to the I-II loop, can also enter into competition with these molecular interactions. It would be via these competitions that the Gβγ complex would cause the regulation of the calcium channel (reduction of the amplitude of the calcium current and slowing down of the activation kinetics).

Thus, one of the main objects of the present invention is to develop a screening test aimed at identifying molecules capable of disturbing the intramolecular interactions and the ionic activity of the α₁ sub-unit of the high-threshold calcium channels, said molecules being capable of being used in particular in the treatment of cerebral ischaemia or neurodegeneration.

A subject of the present invention is the use:
of peptide fragments of the α1 sub-unit of the calcium channels of mammals, said fragments corresponding to the I-II loop and/or to the III-IV loop of said α1 sub-unit, or corresponding to a peptide sequence derived from this I-II or III-IV loop, in particular by substitution, and/or deletion, and/or addition of one or more amino acids, or corresponding to a peptide part of said I-II, or III-IV loop, or of a sequence derived from the latter, in particular a peptide part with at least approximately 5 amino acids, said derived sequence and said part of the I-II loop having the property of said I-II loop of binding to the β sub-unit and to the III-IV loop of said calcium channels, said derived sequence and said part of the III-IV loop having the property of said III-IV loop of binding to said I-II loop,
or of mutated peptide sequences, derived by mutation of one or more amino acids of said abovementioned peptide fragments of the I-II loop of the α1 sub-unit of the calcium channels to the extent that the mutation or mutations in question affect essential amino acids within the context of the expression of the calcium channels at the membrane surface, and/or of peptide sequences derived by mutation of one or more amino acids of said abovementioned peptide fragments of the III-IV loop of the α1 sub-unit of the calcium channels, to the extent that the mutation or mutations in question affect essential amino acids within the context of the inactivation of the calcium channels,
or of cells transformed by nucleotide sequences coding for the abovementioned peptide fragments of the I-II loop and/or of the III-IV loop of the α1 sub-unit of the calcium channels, or coding for the abovementioned mutated peptide sequences,
for the implementation of processes for screening:
molecules restoring the number of calcium channels to normal in cell membranes where this number has abnormally reduced, namely screening β-like molecules capable of being used in the treatment of pathologies linked to an abnormal reduction in the number of calcium channels such as epilepsy, or neuronal degeneration,
and/or molecules increasing the number of calcium channels in the cell membranes, namely screening β-like molecules capable of being used in the treatment of pathologies against which an increase in the number of calcium channels in the plasma membrane would have a beneficial effect, such as Parkinson's disease, insulin-dependent diabetes, or Lambert-Eaton myasthenic syndrome,
and/or molecules restoring the number of calcium channels to normal in the cell membranes where this number has abnormally increased, namely screening molecules capable of being used in the treatment of pathologies linked to an abnormal increase in the number of calcium channels such as cardiac hypertrophy,
and/or molecules reducing the number of calcium channels in the cell membranes, namely screening molecules capable of being used in the treatment of pathologies against which a reduction in the number of calcium channels in the plasma membrane would have a beneficial effect, such as epilepsy, hypertension, angina pectoris, or cerebral ischaemia,
and/or molecules regulating the state of inactivation of the neuronal calcium channels involved in the release of the neurotransmitters, namely screening molecules capable of being used in the stimulation or inhibition of neuronal communication, in particular in the treatment of pathologies against which a regulation of the state of inactivation of the neuronal calcium channels involved in the release of the neurotransmitters would have a beneficial effect, such as epilepsy, ataxia, migraine, Parkinson's disease, or cerebral ischaemia.

A more particular subject of the invention is the abovementioned use of peptide fragments corresponding to the I-II loop of the α1 sub-unit, or to a derived peptide sequence, or to a part of this I-II loop, or of cells transformed by nucleotide sequences coding for said fragments, as defined above, for the implementation of processes for screening:
molecules restoring the number of calcium channels to normal in the cell membranes where this number has abnormally reduced, as defined above,
and/or molecules increasing the number of calcium channels in the cell membranes, as defined above.

The invention relates more particularly to the abovementioned use:

of the peptide sequence corresponding to the I-II loop of the Cav2.1 sub-unit of the calcium channels of rabbit neuronal cells, said sequence corresponding to the following sequence SEQ ID NO: 2:

SGEFAKERERVENRRAFLKLRRQQQIERELNGYMEWISKAEEVILAEDET
DVEQRHPFDGALRRATIKKSKTDLLHPEEAEDQLADIASVGSPFARASIK
SAKLENSSFFHKKERRMRFYIRRMVKTQ

or of the peptide sequence corresponding to the I-II loop of the Cav2.1 sub-unit of the calcium channels of human neuronal cells, said sequence corresponding to the following sequence SEQ ID NO: 4:

SGEFAKERERVENRRAFLKLRRQQQIERELNGYMEWISKAEEVILAEDET
DGEQRHPFDGALRRTTIKKSKTDLLNPEEAEDQLADIASVGSPFARASIK
SAKLENSTFFHKKERRMRFYIRRMVKTQ

or of fragments of the abovementioned sequences SEQ ID NO: 2 and SEQ ID NO: 4, comprising at least the following sequence SEQ ID NO: 15:

QQIERELNGYMEWISKAE

or of cells transformed with the following nucleotide sequences SEQ ID NO: 1 and SEQ ID NO: 3 coding respectively for the abovementioned peptide sequences SEQ ID NO: 2 and SEQ ID NO: 4, or transformed with the nucleotide sequence comprised in the sequences SEQ ID NO: 1 and 3 and coding for the abovementioned sequence SEQ ID NO: 15:

| | |
|---|---|
| tcaggggagtttgccaaagaaagggagcgggtggagaaccggcgcgcattcctgaagctgcggcggcagc | SEQ ID NO: 1 |
| agcagattgaacgcgagctcaacgggtacatggagtggatctcaaaagcagaagaggtgatcctcgcaga | |
| ggacgagaccgacgtggagcagagacatccctttgatggagctctgcggagagccactatcaagaagagc | |
| aagacggacctgctccacccagaggaggcggaggatcagctggccgacatcgcctccgtggggtctccct | |
| ttgcccgagccagcattaaaagtgccaagctggagaactcgagttttttccacaaaaaagagaggagaat | |
| gcgtttctacatccgtcgcatggtcaaaactcag: | |
| tcagggg agtttgccaa agaaagggaa cgggtggaga accggcgggc ttttctgaag | SEQ ID NO: 3 |
| ctgaggcggc aacaacagat tgaacgtgag ctcaatgggt acatggaatg gatctcaaaa | |
| gcagaagagg tgatcctcgc cgaggatgaa actgacgggg agcagaggca tccctttgat | |
| ggagctctgc ggagaaccac cataaagaaa agcaagacag atttgctcaa ccccgaagag | |
| gctgaggatc agctggctga tatagcctct gtgggttctc ccttcgcccg agccagcatt | |
| aaaagtgcca agctggagaa ctcgaccttt tttcacaaaa aggagaggag gatgcgtttc | |
| tacatccgcc gcatggtcaa aactcag | |

Preferably, in the case of the abovementioned use of peptide fragments of the α1 sub-unit having a size larger than approximately 5 amino acids, said fragments are fused on the N-terminal side to a transmembrane peptide sequence, namely a peptide sequence having the effect of maintaining said peptide fragments in the cell membrane, such as the transmembrane sequence of the α chain of the human CD8 receptor contained in the following sequence SEQ ID NO: 5:

LDFACDIYIWAPLAGTCGVLLLSLVITLYCNHR

As a variant, the invention also relates to the abovementioned use of cells transformed with an exogenous recombinant nucleotide sequence coding for a transmembrane peptide sequence as defined above, this last sequence being situated upstream of the sequence coding for the abovementioned peptide fragment of the α1 sub-unit.

A subject of the invention is also any process for screening molecules restoring the number of calcium channels to normal in the cell membranes where this number has abnormally reduced, as defined above, and/or of molecules increasing the number of calcium channels in the cell membranes, as defined above, characterized in that it comprises the following stages:
bringing together peptide fragments of the α1 sub-unit as defined above and cells expressing calcium channels for a time sufficient for the number of calcium channels to be reduced significantly at the surface of said cells, then with the molecules to be tested,
or bringing together cells transformed using nucleotide sequences coding for peptide fragments of the α1 sub-unit as defined above, thus reducing the number of calcium channels at the surface of said cells, and the molecules to be tested,
detection of any increase in the number of calcium channels at the surface of the transformed cells proving the effect of the molecules tested of increasing the number of calcium channels at the surface of the cell membranes, in particular by electrophysiological measurements, or using fluorescent or radioactive probes.

A more particular subject of the invention is the abovementioned use of peptide sequences derived by mutation of one or more amino acids of the peptide fragments of the I-II loop of the α1 sub-unit of the calcium channels, the mutation or mutations in question affecting essential amino acids within the context of the expression of the calcium channels at the membrane surface, to the extent that their mutation has the effect of increasing or reducing the expression at the surface of the plasma membrane of the calcium channels, for the implementation of processes for screening:
molecules restoring the number of calcium channels to normal in the cell membranes where this number has abnormally reduced, as defined above,
and/or molecules increasing the number of calcium channels in the cell membranes, as defined above,
and/or molecules restoring the number of calcium channels to normal in the cell membranes where this number has abnormally increased, as defined above,
and/or molecules reducing the number of calcium channels in the cell membranes, as defined in claim 1.

The invention relates more particularly to the abovementioned use of peptide sequences comprising one or more mutations having the effect of increasing the expression at the surface of the plasma membrane of calcium channels, said peptide sequences being derived by mutation of at least one of the amino acids situated in positions 383, 395, 396, 398, 427, and 428 of the Cav2.1 sub-unit of the calcium channels of rabbit or human neuronal cells, namely of the peptide sequences corresponding to the sequence SEQ ID NO: 2 or SEQ ID NO: 4, in which at least one of Q in position 24, W in position 36, I in position 37, K in position 39, K in position 68, and K in position 69, is substituted by a natural or non-natural amino acid, in particular by an alanine.

The invention relates more particularly to the abovementioned use of peptide sequences comprising one or more mutations having the effect of reducing the expression at the surface of the plasma membrane of the calcium channels, said peptide sequences being derived by mutation of at least one of the amino acids situated in positions 387, 422, and 423 of the Cav2.1 sub-unit of the calcium channels of rabbit neuronal cells, or to the equivalent positions of the human Cav2.1 sub-unit, namely of the peptide sequences corresponding to the sequence SEQ ID NO: 2 or SEQ ID NO: 4, in which at least one of R in position 28, R in position 63, and R in position 64, is substituted by a natural or non-natural amino acid, in particular by an alanine.

The invention also relates to any process for screening:
molecules restoring the number of calcium channels to normal in the cell membranes where this number has abnormally reduced, as defined above,
and/or molecules increasing the number of calcium channels in the cell membranes, as defined above,
and/or molecules restoring the number of calcium channels to normal in the cell membranes where this number has abnormally increased, as defined in claim 1,
and/or molecules reducing the number of calcium channels in the cell membranes, as defined above,
characterized in that it comprises the following stages:
bringing together peptide sequences derived from the I-II loop of the α1 sub-unit as defined above and molecules to be tested already selected for their ability to bind specifically to the non-mutated peptide sequences corresponding to said I-II loops,
selection of the abovementioned molecules binding specifically to the I-II loops and not binding to the abovementioned derived peptide sequences, in particular by the use of the Biacore technique (O'Shannessy D J et al. (1992) “Immobilization chemistries suitable for use in the Biacore surface plasmon resonance detector” Anal Biochem. 205, 132-136) based on a physical and optical principle of molecular interaction,
if appropriate, bringing together the molecules selected in the previous stage and cells expressing calcium channels, and observation of any effect of the molecules selected on the increase or reduction in the number of calcium channels at the surface of said cells, in particular by electrophysiological measurements, or using fluorescent or radioactive probes.

A subject of the invention is also the abovementioned use of peptide sequences derived by mutation of one or more amino acids of the peptide fragments of the I-II loop of the α1 sub-unit of the calcium channels, the mutation or mutations in question affecting essential amino acids within the context of the inactivation of the calcium channels present at the membrane surface, to the extent that their mutation has the effect of modulating the activity of the calcium channels, for the implementation of processes for screening molecules regulating the state of inactivation of the neuronal calcium channels involved in the release of the neurotransmitters, as defined above.

A more particular subject of the invention is the abovementioned use of peptide sequences comprising one or more mutations having the effect of inactivating the calcium channels, said peptide sequences being derived by mutation of at least one of the amino acids situated in positions 387 and 388, and more particularly by the single mutation of the amino acid situated in position 388, of the Cav2.1 sub-unit of the calcium channels of rabbit or human neuronal cells, namely of the peptide sequences corresponding to the sequence SEQ ID NO: 2 or SEQ ID NO: 4, in which at least one of R in position 28, and E in position 29, substituted by a natural or non-natural amino acid, in particular R28 is substituted by an alanine or by E, and E29 is substituted by an alanine.

The invention also relates to any process for screening molecules regulating the state of inactivation of the neuronal calcium channels involved in the release of the neurotransmitters, as defined above, characterized in that it comprises the following stages:
bringing together peptide sequences derived from the I-II loop of the α1 sub-unit as defined above and molecules to be tested already selected for their ability to bind specifically to the non-mutated peptide sequences corresponding to said I-II loops,
selection of the abovementioned molecules binding specifically to the I-II loops and not binding to the abovementioned derived peptide sequences, in particular by the abovementioned Biacore technique,
if appropriate, bringing together the molecules selected in the previous stage and cells expressing calcium channels, and observation of any effect of the molecules selected on the regulation of the state of inactivation of the neuronal calcium channels involved in the release of the neurotransmitters from said cells, in particular by electrophysiological measurements.

A subject of the invention is also the abovementioned use of peptide fragments corresponding to the III-IV loop of the α1 sub-unit, or to a derived peptide sequence, or to a part of this III-IV loop, or of cells transformed by nucleotide sequences coding for said fragments, as defined above, for the implementation of processes for screening molecules regulating the state of inactivation of the neuronal calcium channels involved in the release of the neurotransmitters, as defined above.

The invention has more particularly the abovementioned use:

of the peptide sequence corresponding to the III-IV loop of the Cav2.1 sub-unit of the calcium channels of human or rabbit neuronal cells, said sequence corresponding to the following sequence SEQ ID NO: 7:

ITFQEQGDKMMEEYSLEKNERACIDFAISAKPLTRHMPQNKQSFQYRMWQ
FVVSP

or of the cells transformed with the nucleotide sequence coding for the III-IV loop of the Cav2.1 sub-unit of the calcium channels of human neuronal cells, said sequence corresponding to the following sequence SEQ ID NO: 6:

at caccttccag gagcaagggg acaagatgat ggaggaatac agcctggaga aaaatgagag
ggcctgcatt gatttcgcca tcagcgccaa gccgctgacc cgacacatgc cgcagaacaa
gcagagcttc cagtaccgca tgtggcagtt cgtggtgtct ccg

or of cells transformed with the nucleotide sequence SEQ ID NO: 6 and SEQ ID NO: 8 coding respectively for the abovementioned peptide sequences SEQ ID NO: 7 and SEQ ID NO: 9 coding for the III-IV loop of the Cav2.1 sub-unit of the calcium channels of rabbit neuronal cells, said sequence corresponding to the following sequence SEQ ID NO: 8:

atcacct tccaggagca gggcgacaag atgatggagg agtacagctt ggagaaaaac
gagagggcct gcatcgactt cgccatcagt gccaagccgc tgaccaggca catgccccag
aacaagcaga gcttccagta ccgcatgtgg cagttcgtgg tgtccccg

Preferably in the case of the use of peptide fragments of the α1 sub-unit having a size larger than approximately 5 amino acids, said fragments are fused or not fused on the N-terminal side to a transmembrane peptide sequence, namely a peptide sequence having the effect of maintaining said peptide fragments in the cell membrane, such as the transmembrane sequence of the α chain of the human CD8 receptor contained in the following sequence SEQ ID NO: 5:

LDFACDIYIWAPLAGTCGVLLLSLVITLYCNHR

As a variant, the invention also relates to the abovementioned use of cells transformed with an exogenous recombinant nucleotide sequence coding for a transmembrane peptide sequence as defined above, this last sequence being situated upstream of the sequence coding for the abovementioned peptide fragment of the α1 sub-unit.

A subject of the invention is also any process for screening molecules regulating the state of inactivation of the neuronal calcium channels involved in the release of the neurotransmitters, as defined above, characterized in that it comprises the following stages:
bringing together peptide fragments of the α1 sub-unit as above and cells expressing calcium channels for a time sufficient for the state of inactivation of the channels to be modified, then with the molecules to be tested,
or bringing together cells transformed using nucleotide sequences coding for peptide fragments of the α1 sub-unit as defined above and the molecules to be tested,
detection of the effect of the molecules tested on the state of inactivation of the neuronal calcium channels involved in the release of the neurotransmitters, in particular by electrophysiological measurements.

The invention also relates to the abovementioned use of peptide sequences derived by mutation of one or more amino acids of the peptide fragments of the III-IV loop of the α1 sub-unit of the calcium channels, the mutation or mutations in question affecting essential amino acids within the context of the inactivation of the calcium channels present at the membrane surface, to the extent that their mutation has the effect of modulating the activity of the calcium channels, for the implementation of processes for screening molecules regulating the state of inactivation of the neuronal calcium channels involved in the release of the neurotransmitters, as defined above.

A more particular subject of the invention is the abovementioned use of peptide sequences comprising one or more mutations having the effect of inactivating the calcium channels, said peptide sequences being derived by mutation of at least one of the amino acids of the peptide sequence corresponding to the sequence SEQ ID NO: 7.

The invention therefore relates more particularly to the abovementioned use of peptide sequences comprising one or more mutations having the effect of inactivating the calcium channels, said peptide sequences being derived by mutation of at least one of the amino acids situated between the positions 8 and 19 of the peptide sequence corresponding to the sequence SEQ ID NO: 7, namely at least one of the amino acids included in the sequence: DKMMEEYSLEKN.

The invention also relates to any process for screening molecules regulating the state of inactivation of the neuronal calcium channels involved in the release of the neurotransmitters, as defined above, characterized in that it comprises the following stages:
bringing together peptide sequences derived from the III-IV loop of the α1 sub-unit as defined above and the molecules to be tested already selected for their ability to bind specifically to the non-mutated peptide sequences corresponding to said III-IV loops,
selection of the abovementioned molecules binding specifically to the III-IV loops and not binding to the abovementioned derived peptide sequences, in particular according to the abovementioned Biacore technique,
if appropriate, bringing together the molecules selected in the previous stage and cells expressing calcium channels, and observation of any effect of the molecules selected on the regulation of the state of inactivation of the neuronal calcium channels involved in the release of the neurotransmitters from said cells, in particular by electrophysiological measurements.

A subject of the invention is also the following peptide sequences:
the sequences SEQ ID NO: 2 and SEQ ID NO: 4 in which at least one of Q in position 24, W in position 36, I in position 37, K in position 39, K in position 68, and K in position 69, is substituted by a natural or non-natural amino acid, in particular by an alanine,
the sequences SEQ ID NO: 2 and SEQ ID NO: 4 in which at least one of R in position 28, R in position 63, and R in position 64, is substituted by a natural or non-natural amino acid, in particular by an alanine,
the sequences SEQ ID NO: 2 and SEQ ID NO: 4 in which at least one of R in position 28, and E in position 29, is substituted by a natural or non-natural amino acid, in particular R28 is substituted by an alanine or by E, and E29 is substituted by an alanine,
the following sequence SEQ ID NO: 15: QQIERELNGYMEWISKAE.

The invention is further illustrated using the experimental results which follow:

I—Demonstration of the Reduction in the Number of Calcium Channels at the Membrane Surface of Bovine Chromaffin Cells and Rat Cerebellum Granular Cells.

The Inventors' results show that the expression of the I-II loop of the Cav2.1 sub-unit in bovine chromaffin cells or rat cerebellum granular cells induces an appreciable reduction in the high threshold calcium channels at the membrane surface of these cells. The measurements carried out show that more than 50% of the high-threshold calcium current is reduced in these cells following the expression of this sequence.

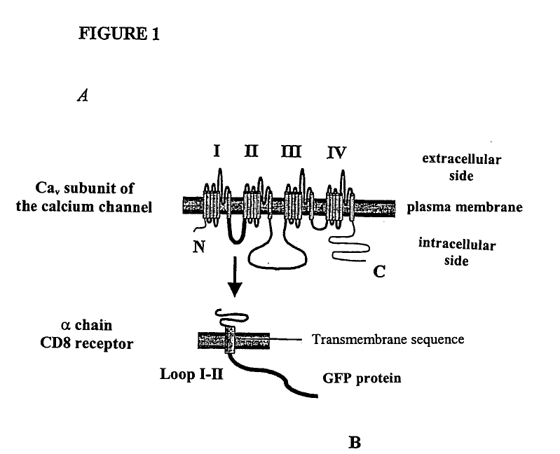

The expression of the I-II loop of the Caᵥ2.1 channel is induced following a cell transfection method (thanks to a transfection agent: lipofectamine, fugene etc.). In order to have the ability to bind the endogenous β sub-units to these cells, we have noted that the ability of this I-II sequence to reduce the expression at the plasma membrane of the endogenous high-threshold calcium channels is facilitated by its coupling to a transmembrane segment. The Inventors have used for their experiments the transmembrane segment of the α chain of the human CD8 receptor, coupled to the amino-terminal part of the I-II loop. In order to facilitate the detection of the transfected cells, the carboxy-terminal part of the I-II loop was coupled to GFP protein (Green Fluorescent Protein) which has the property of green fluorescence (easily detectable in fluorescence microscopy). FIG. 1A illustrates the construction used for these experiments. FIG. 1B illustrates the amino-acid sequence of the transmembrane sequence of the α chain of the CD8 receptor.

Transmembrane sequence of the α chain of the human CD8 receptor (contained in): LDFACDIYIWAPLAGTCGVLLLSLVITLYCNHR

II—Demonstration of the Importance of the Amino Acids Arg 387 and Glu 388 of the Cav2.1 Sequence of the Rabbit in the Inactivation of this Channel.

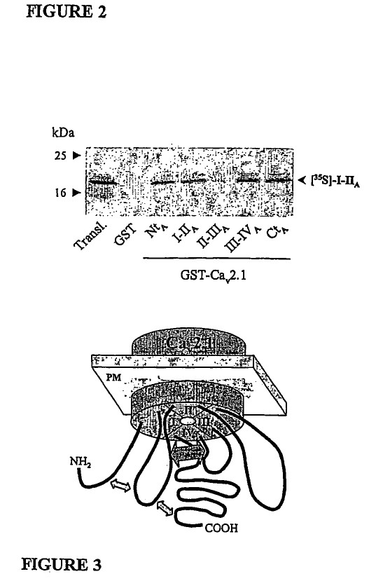

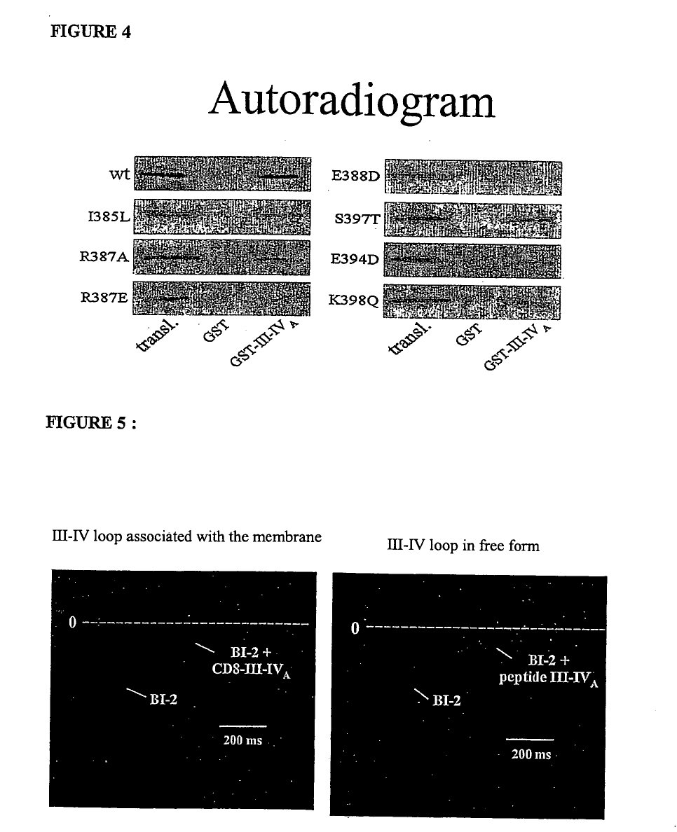

The experimental results obtained demonstrate that the amino acids of the I-II loop of the Caᵥ2.1 calcium channel are involved in the inactivation of this channel. Two modes of participation are possible:
1—In the absence of β sub-unit, these two amino acids are involved in a molecular interaction with the III-IV loop of the Cav2.1 sub-unit (FIGS. 2 and 3). If these interactions are interrupted, either by mutagenesis of the amino acids Arg 387 or Glu 388 (FIG. 4), or by expressing the III-IV loop (which can then enter into competition with the natural interaction between the I-II and III-IV loops of the Caᵥ2.1 channel; FIG. 5), the inactivation of the calcium channel is facilitated. A facilitated inactivation represents, either an accelerated transition from the open state of the channel towards its inactivated state, or a reduction in the number of channels which can be activated at a given membrane voltage of the cell. The methodologies allowing the measurement of the calcium currents and the analysis of the inactivation are given in Annex A.
2—In the presence of β sub-unit (the normal configuration of the channel at the plasma membrane), the electric charge carried by the radical chains of these amino acids (+charge for Arg 387 and−charge for Glu 388) plays a role in the inactivation of this channel.

The results show that in these two hypothetical cases, these amino acids can be taken as the target of a direct pharmacological intervention in order to modulate the inactivation of the calcium channels. The presence of these amino acids in most of the neuronal calcium channels (Caᵥ2.1, Caᵥ2.2 and Caᵥ2.3), and in most species, including humans, strongly suggests that these amino acids are also involved in the inactivation of these channels.

FIG. 2: Binding of the I-II loop of Caᵥ2.1 to different intracellular loops. A binding of I-II is evident on the III-IV loop of Caᵥ2.1. This III-IV loop has been expressed and purified as a GST (Glutathione-S-Tranferase) fusion protein. The I-II protein of the channel has been translated in vitro and labelled by a radioactive amino acid (³⁵S-methionine) in order to monitor its binding to the GST-III-IV fusion protein.

FIG. 3: Diagrammatic representation showing the intramolecular interactions between the different loops of the Caᵥ2.1 channel. The green arrow illustrates the interaction between the I-II loop and the III-IV loop of this channel. PM=plasma membrane.

FIG. 4: Demonstration of the loss of binding of the I-II loop of the Cav2.1 channel to the GST-III-IV fusion protein following the mutation of the amino acids Arg 387 or Glu 388. These two amino acids are therefore indispensable to the binding of the I-II loop to the III-IV loop in the absence of the β sub-unit of the calcium channel.

FIG. 5: The expression of the III-IV loop accelerates the inactivation kinetics of the Cav2.1 channel. This acceleration is similar if the III-IV loop expressed is associated with the plasma membrane (left, via a coupling in the amino-terminal position with the α chain of the human CD8 receptor) or is free in the cytoplasm (right, injection of a peptide representing the 40 carboxy-terminal amino acids of the III-IV loop).

III—Demonstration of the Importance of Several Amino Acids of the I-II Loop of the Caᵥ2.1 Sequence of the Rabbit in the Retention of this Channel at the Level of the Endoplasmic Reticulum.

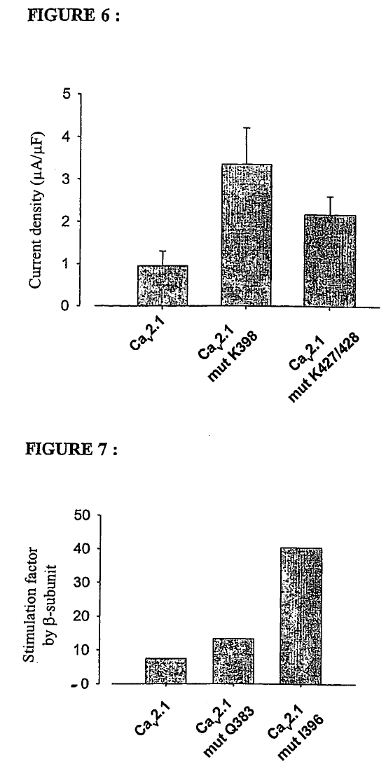

The results of mutagenesis of the I-II loop of the Cav2.1 sub-unit and of the expression in the Xenopus oocyte of the corresponding mutants show that a certain number of amino acids of the I-II loop can be chosen as a target in order to increase the membrane addressing of the high-threshold calcium channels. We can classify these amino acids into two categories: (i) the amino acids the function of which is independent of the β sub-unit of the calcium channels and the mutation of which induces an increase in expression of the calcium channels at the surface of the membrane, and (ii) the amino acids the mutation alone of which does not contribute to a facilitated surface expression of the Caᵥ2.1 channel when it is expressed alone, but rather to a facilitation of the action of the β sub-units. The action of said β sub-units being precisely to facilitate the expression of the high-threshold calcium channels, the expression at the cell surface of the whole of the Caᵥ2.1/β complex is also facilitated by the mutagenesis of these residues. The importance of the β-independent amino acids is shown in FIG. 6, whilst that of the β-dependent residues is illustrated in FIG. 7. FIG. 8 recapitulates the position of the amino acids of the I-II loop the mutation of which can facilitate the expression of the calcium channels.

FIG. 6: Expression of mutated Caᵥ2.1 sub-units increasing the expression of the channel (in the absence of β sub-unit). Measurement in current density at the surface of the plasma membrane of the Xenopus oocytes. See Annex A hereafter for the methodology of expression and recording of the electric currents.

FIG. 7: Expression of mutated Caᵥ2.1 sub-units facilitating the action of stimulation of expression of the membrane surface of the calcium channels by the β sub-unit. See Annex A for the methodology of expression and recording of the electric currents.

Hereafter are represented the positions of the amino acids, the mutation of which favours the expression at the surface of the plasma membrane of the Caᵥ2.1 channel. In bold, β-independent amino acids, and underlined, the β-dependent amino acids.

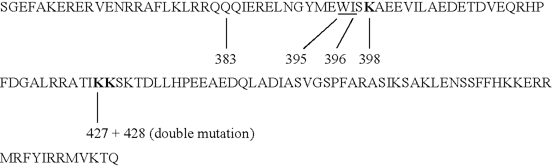

It is also possible to identify the position of the amino acids, the mutation of which leads to a reduction in the expression at the surface of the cells. This information is given by way of example hereafter.

Position of the amino acids, the mutation of which leads to an appreciable reduction of expression of the Caᵥ2.1 channel at the membrane (in the presence and absence of β sub-unit).

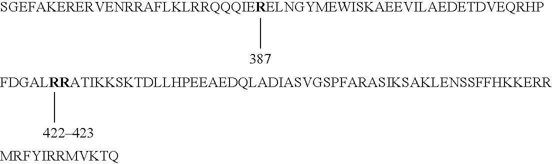

In order to extend the validity of our results, we have aligned the sequence of the I-II loop of the rabbit Caᵥ2.1 channel to that of the I-II sequence of the human Caᵥ2.1 channel. The positions of the amino acids essential to all the functions mentioned are indicated in bold hereafter.

Alignment of the rabbit and human Caᵥ2.1 sequences. In bold, essential amino acids. Underlined: the amino acids which differ between the two sequences.

```
          Cav2.1 human
SGEFAKERERVENRRAFLKLRRQQQIERELNGYMEWISKAEEVIL
          Cav2.1 rabbit
SGEFAKERERVENRRAFLKLRRQQQIERELNGYMEWISKAEEVIL
AEDETDGEQRHPFDGALRRTTIKKSKTDLLNPEEAEDQLADIASVGSPFARASIKS
AEDETDVEQRHPFDGALRRATIKKSKTDLLHPEEAEDQLADIASVGSPFARASIKS
AKLENSTFFHKKERRMRFYIRRMVKTQ
AKLENSSFFHKKERRMRFYIRRMVKTQ
```

### Annex A

Expression of the Caᵥ2.1 Channel in the Xenopus Oocyte, Electrophysiological Measurement of the Calcium Currents and Analysis of the Inactivation Properties of the Calcium Channel.

Expression

Xenopus oocytes are removed at Stages V and VI from ovaries of Xenopus laevis frogs from South Africa and maintained in standard saline solution (Barth's medium). The cells are treated with collagenase type IA (2 mg/ml) for two hours and the follicular membranes are eliminated manually. The oocytes are maintained in DNOM medium (defined nutrient oocyte medium) for 2 days before injection of cRNA coding for the Cav2.1 channel. The injection of this cRNA coding for the wild-type or mutated channel is carried out at a concentration of 0.3 μg/μl. In the case of coinjection of other cRNAs (for example: CD8-III-IV, III-IV, etc.), these cRNAs are injected at a concentration of 0.1 μg/μl. The injections of III-IV peptide are carried out at a final intracellular concentration of 10 μM. In all hypothetical cases, the injection volume does not exceed 50 nl per oocyte. Following the cRNA injections, the cells are kept at 16° C. in DNOM medium for a minimum of 4-5 days before continuing with the electrophysiological recordings.

Electrophysiological Measurements

We applied the two-electrode voltage-clamp technique for the recordings of Ba²⁺ currents (the Ba²+ is more permeable than the Ca²⁺ through the high-threshold calcium channels and facilitates our analyses). The recordings are carried out with a GeneClamp amplifier from Axon Instruments (Foster City, Calif.). The extracellular recording medium contains (in mM): Ba(OH)₂ 40, NaOH 50, KCl 3, HEPES 5, niflumic acid 1, pH 7.4 with methane sulphonic acid. The electrodes are filled with (in mM) KCl 140, EGTA 10 and HEPES 10 (pH 7.2 with KOH) and have electric resistances comprised between 0.5 and 1 MΩ. The electric current recordings are filtered “on-line” at 2 kHz, the leakage current is subtracted by a protocol P/6, and sampled at 5-10 kHz. The data are analyzed with the pCLAMP software suite version 6.03 (Axon Instruments).

Analysis of the Inactivation Properties of the Caᵥ2.1 Channel

In order to establish inactivation curves as a function of the cell membrane voltage, the oocytes are depolarized for at least 30 seconds at a given voltage value (between −100 and 10 mV; maintenance voltage), then the calcium current is triggered by a cell depolarization at +20 mV. This procedure is repeated several times for increasing maintenance voltage values (from 10 mV in 10 mV steps). The maximum amplitude of the Ba²⁺ currents thus obtained for each depolarization at +20 mV is compared to the amplitude of the maximum current obtained by a depolarization starting from the voltage of −100 mV to +20 mV (reference value corresponding to 0% of inactivation of the channels). The inactivation of the calcium channels is also triggered by a depolarization maintained at +20 mV (starting from a maintenance value of −100 mV). The decreasing current kinetics illustrate the process of inactivation of the calcium channels which is triggered by the depolarization at +20 mV. In order to compare the differences in inactivation kinetics, we chose the time necessary for semi-inactivation of the calcium channels as a reference value.

1-21. (canceled)

22. A screening test for identifying molecules capable of disturbing the intramolecular interactions and the ionic activity of a α1 sub-unit from a calcium channel of a mammal, comprising:
peptide fragments of a α1 sub-unit from a calcium channel of a mammal, said fragments corresponding to the I-II loop and/or to the III-IV loop of said α1 sub-unit, or corresponding to a peptide sequence derived from this I-II or III-IV loop, by substitution, and/or deletion, and/or addition of one or more amino acids, or corresponding to a peptide part of said I-II, or III-IV loop, or of a sequence derived from the latter, said derived sequence and said part of the I-II loop having the property of said I-II loop of binding to a β sub-unit and to the III-IV loop of said calcium channel, said derived sequence and said part of the III-IV loop having the property of said III-IV loop of binding to said I-II loop,
or of mutated peptide sequences, derived by mutation of one or more amino acids of said peptide fragments of the I-II loop of the α1 sub-unit of the calcium channels to the extent that the mutation or mutations in question affect essential amino acids within the context of the expression of the calcium channels at the membrane surface, and/or of peptide sequences derived by mutation of one or more amino acids of said peptide fragments of the III-IV loop of the α1 sub-unit of the calcium channels, to the extent that the mutation or mutations in question affect essential amino acids within the context of the inactivation of the calcium channels,
or of cells transformed by nucleotide sequences coding for said peptide fragments of the I-II loop and/or of the III-IV loop of the α1 sub-unit of the calcium channels, or coding for said mutated peptide sequences,
for the implementation of a process for screening
molecules restoring the number of calcium channels to normal in cell membranes where this number has abnormally reduced, namely screening β-like molecules capable of being used in the treatment of pathologies linked to an abnormal reduction in the number of calcium channels such as epilepsy, or neuronal degeneration,
and/or molecules increasing the number of calcium channels in the cell membranes, namely screening β-like molecules capable of being used in the treatment of pathologies against which an increase in the number of calcium channels in the plasma membrane would have a beneficial effect, such as Parkinson's disease, insulin-dependent diabetes, or Lambert-Eaton myasthenic syndrome,
and/or molecules restoring the number of calcium channels to normal in the cell membranes where this number has abnormally increased, namely screening molecules capable of being used in the treatment of pathologies linked to an abnormal increase in the number of calcium channels such as cardiac hypertrophy,
and/or molecules reducing the number of calcium channels in the cell membranes, namely screening molecules capable of being used in the treatment of pathologies against which a reduction in the number of calcium channels in the plasma membrane would have a beneficial effect, such as epilepsy, hypertension, angina pectoris, or cerebral ischaemia,
and/or molecules regulating the state of inactivation of the neuronal calcium channels involved in the release of neurotransmitters, namely screening molecules capable of being used in the stimulation or inhibition of neuronal communication, in particular in the treatment of pathologies against which a regulation of the state of inactivation of the neuronal calcium channels involved in the release of neurotransmitters would have a beneficial effect, such as epilepsy, ataxia, migraine, Parkinson's disease, or cerebral ischaemia.

23. The screening test according to claim 22, wherein the screening test comprises
a peptide sequence corresponding to the I-II loop of the Cav2.1 sub-unit of the calcium channels of rabbit neuronal cells, said sequence corresponding to the following sequence SEQ ID NO: 2:
SGEFAKERERVENRRAFLKLRRQQQIERELNGYMEWISKAEEVI
LAEDETDVEQRHPFDGALRRATIKKSKTDLLHPEEAEDQLADIASVGSPF
ARASIKSAKLENSSFFHKKERRMRFYIRRMVKTQ
or a peptide sequence corresponding to the I-II loop of the Cav2.1 sub-unit of the calcium channels of human neuronal cells, said sequence corresponding to the following sequence SEQ ID NO: 4:
SGEFAKERERVENRRAFLKLRRQQQIERELNGYMEWISKAEEVI
LAEDETDGEQRHPFDGALRRTTIKKSKTDLLNPEEAEDQLADIASVGSPF
ARASIKSAKLENSTFFHKKERRMRFYIRRMVKTQ
or a fragment of said sequences SEQ ID NO: 2 and SEQ ID NO: 4, comprising at least the following sequence SEQ ID NO: 15: QQIERELNGYMEWISKAE,
or of cells transformed with the following nucleotide sequences SEQ ID NO: 1 and SEQ ID NO: 3 coding respectively for said peptide sequences SEQ ID NO: 2 and SEQ ID NO: 4, or transformed with the nucleotide sequence comprised in the sequences SEQ ID NO: 1 and 3 and coding for said sequence SEQ ID NO: 15:

| | |
|---|---|
| tcaggggagtttgccaaagaaagggagcgggtggagaaccggcgcgcattcctgaagctgcggcggc | SEQ ID NO: 1 |
| agcagcagattgaacgcgagctcaacgggtacatggagtggatctcaaaagcagaagaggtgatcctcgcag | |
| aggacgagaccgacgtggagcagagacatccctttgatggagctctgcggagagccactatcaagaagagca | |
| agacggacctgctccacccagaggaggcggaggatcagctggccgacatcgcctccgtggggtctccctttg | |
| cccgagccagcattaaaagtgccaagctggagaactcgagttttttccacaaaaaagagaggagaatgcgtt | |
| tctacatccgtcgcatggtcaaaactcag: | |
| tcagggg agtttgccaa agaaagggaa cgggtggaga accggcgggc ttttctgaag | SEQ ID N^{o}: 3 |
| ctgaggcggc aacaacagat tgaacgtgag ctcaatgggt acatggaatg gatctcaaaa | |
| gcagaagagg tgatcctcgc cgaggatgaa actgacgggg agcagaggca tccctttgat | |
| ggagctctgc ggagaaccac cataaagaaa agcaagacag atttgctcaa ccccgaagag | |
| gctgaggatc agctggctga tatagcctct gtgggttctc ccttcgcccg agccagcatt | |
| aaaagtgcca agctggagaa ctcgaccttt tttcacaaaa aggagaggag gatgcgtttc | |
| tacatccgcc gcatggtcaa aactcag: | |

24. The screening test according to claim 22, wherein said fragments are fused on the N-terminal side to a transmembrane peptide sequence, namely a peptide sequence having the effect of maintaining said peptide fragments in the cell membrane, such as the transmembrane sequence of the α chain of the human CD8 receptor contained in the following sequence SEQ ID NO: 5:
LDFACDIYIWAPLAGTCGVLLLSLVITLYCNHR
or of cells transformed with an exogenous recombinant nucleotide sequence coding for a transmembrane peptide sequence as defined above, this last sequence being situated upstream of the sequence coding for said peptide fragment of the α1 sub-unit.

25. A process for screening molecules that restore the number of calcium channels to normal in the cell membranes where said number is abnormally reduced, and/or of molecules increasing the number of calcium channels in the cell membranes, characterized in that it comprises the following steps:
bringing together peptide fragments of the α1 sub-unit and cells expressing calcium channels for a time sufficient for the number of calcium channels to be reduced significantly at the surface of said cells, then with the molecules to be tested, or bringing together cells transformed using nucleotide sequences coding for peptide fragments of the α1 sub-unit, thus reducing the number of calcium channels at the surface of said cells and the molecules to be tested, and
detecting any increase or decrease in the number of calcium channels at the surface of the transformed cells proving the effect of the molecules tested of increasing the number of calcium channels at the surface of the cell membranes.

26. The screening test according to claim 22, wherein said peptide sequences are derived by mutation of one or more amino acids of the peptide fragments of the I-II loop of the α1 sub-unit of the calcium channels, the mutation or mutations in question affecting essential amino acids within the context of the expression of the calcium channels at the membrane surface, to the extent that their mutation has the effect of increasing or reducing the expression at the surface of the plasma membrane of the calcium channels, for the implementation of processes for screening:
molecules restoring the number of calcium channels to normal in the cell membranes where this number has abnormally reduced,
and/or molecules increasing the number of calcium channels in the cell membranes,
and/or molecules restoring the number of calcium channels to normal in the cell membranes where this number has abnormally increased,
and/or molecules reducing the number of calcium channels in the cell membranes.

27. The screening test according to claim 26, wherein said peptide sequences comprise one or more mutations having the effect of increasing the expression at the surface of the plasma membrane of calcium channels, said peptide sequences being derived by mutation of at least one of the amino acids situated in positions 383, 395, 396, 398, 427, and 428 of the Cav2.1 sub-unit of the calcium channels of rabbit or human neuronal cells, namely of the peptide sequences (SEQ ID NOS 9 and 10) corresponding to the sequence SEQ ID NO: 2 or SEQ ID NO: 4 respectively, in which at least one of Q in position 24, W in position 36, I in position 37, K in position 39, K in position 68, and K in position 69, is substituted by a natural or non-natural amino acid.

28. The screening test according to claim 26, wherein said peptide sequences comprising one or more mutations having the effect of reducing the expression at the surface of the plasma membrane of the calcium channels, said peptide sequences being derived by mutation of at least one of the amino acids situated in positions 387, 422, and 423 of the Cav2.1 sub-unit of the calcium channels of rabbit, or human, neuronal cells, namely of the peptide sequences (SEQ ID NOS 11 and 12) corresponding to the sequence SEQ ID NO: 2 or SEQ ID NO: 4 respectively, in which at least one of R in position 28, R in position 63, and R in position 64, is substituted by a natural or non-natural amino acid.

29. A process for screening:
molecules restoring the number of calcium channels to normal in the cell membranes where this number has abnormally reduced,
and/or molecules increasing the number of calcium channels in the cell membranes,
and/or molecules restoring the number of calcium channels to normal in the cell membranes where this number has abnormally increased,
and/or molecules reducing the number of calcium channels in the cell membranes, characterized in that it comprises the following steps:
bringing together peptide sequences derived from the I-II loop of the α1 sub-unit according to claim 22 and the molecules to be tested already selected for their ability to bind specifically to the non-mutated peptide sequences corresponding to said I-II loops, selecting said molecules binding specifically to the I-II loops and not binding to said derived peptide sequences, if appropriate, bringing together the molecules selected in the previous stage and cells expressing calcium channels, and observing any effect of the molecules on the increase or reduction in the number of calcium channels at the surface of said cells.

30. The screening test according to claim 22, wherein the peptide sequences are derived by mutation of one or more amino acids of the peptide fragments of the I-II loop of the al subunit of the calcium channels, the mutation or mutations in question affecting essential amino acids within the context of the activity of the calcium channels at the membrane surface, to the extent that their mutation has the effect of modulating the activity of the calcium channels, for the implementation of processes for screening molecules regulating the state of inactivation of the neuronal calcium channels involved in the release of neurotransmitters.

31. The screening test according to claim 30, wherein said peptide sequences comprise one or more mutations having the effect of inactivating the calcium channels, said peptide sequences being derived by mutation of at least one of the amino acids situated in positions 387 and 388 of the Cav2.1 sub-unit of the calcium channels of rabbit or human neuronal cells, namely of the peptide sequences (SEQ ID NOS 13 and 14) corresponding to the sequence SEQ ID NO: 2 or SEQ ID NO: 4 respectively, in which at least one of R in position 28, and E in position 29, is substituted by a natural or non-natural amino acid.

32. A process for screening molecules regulating the state of inactivation of the neuronal calcium channels involved in the release of neurotransmitters, characterized in that it comprises the following steps:
bringing together peptide sequences derived from the I-II loop of the α1 sub-unit according to claim 30 and the molecules to be tested already selected for their ability to bind specifically to the non-mutated peptide sequences corresponding to said I-II loops, selecting said molecules binding specifically to the I-II loops and not binding to said derived peptide sequences, if appropriate, bringing together the molecules selected in a previous stage and cells expressing calcium channels, and observing any effect of the molecules on the regulation of the state of inactivation of the neuronal calcium channels involved in the release of neurotransmitters from said cells.

33. The screening test according to claim 22, wherein said peptide fragments correspond to the III-IV loop of the α1 sub-unit, or to a derived peptide sequence, or to a part of this III-IV loop, or of cells transformed by nucleotide sequences coding for said fragments, for the implementation of processes for screening molecules regulating the state of inactivation of the neuronal calcium channels involved in the release of the neurotransmitters.

34. The screening test according to claim 22, wherein the peptide sequence corresponds to the III-IV loop of the Cav2.1 sub-unit of the calcium channels of human or rabbit neuronal cells, said sequence corresponding to the following sequence SEQ ID NO: 7:
ITFQEQGDKMMEEYSLEKNERACIDFAISAKPLTRHMPQNKQSFQYRMWQ
FVVSP
or of the cells transformed with the nucleotide sequence coding for the III-IV loop of the Cav2.1 sub-unit of the calcium channels of human neuronal cells, said sequence corresponding to the following sequence SEQ ID NO: 6:
at caccttccag gagcaagggg acaagatgat ggaggaatac agcctggaga
aaaatgagag ggcctgcatt gatttcgcca tcagcgccaa gccgctgacc cgacacatgc
cgcagaacaa gcagagcttc cagtaccgca tgtggcagtt cgtggtgtct ccg
or of cells transformed with the nucleotide sequence coding for the III-IV loop of the Cav2.1 sub-unit of the calcium channels of rabbit neuronal cells, said sequence corresponding to the following sequence SEQ ID NO: 8:
atcacct tccaggagca gggcgacaag atgatggagg agtacagctt ggagaaaaac
gagagggcct gcatcgactt cgccatcagt gccaagccgc tgaccaggca catgccccag
aacaagcaga gcttccagta ccgcatgtgg cagttcgtgg tgtccccg

35. The screening test according to claim 22, wherein said fragments are fused on the N-terminal side to a transmembrane peptide sequence, namely a peptide sequence having the effect of maintaining said peptide fragments in the cell membrane, such as the transmembrane sequence of the α chain of the human CD8 receptor contained in the following sequence SEQ ID NO: 5:
LDFACDIYIWAPLAGTCGVLLLSLVITLYCNHR
or of cells transformed with an exogenous recombinant nucleotide sequence coding for a transmembrane peptide sequence as defined above, this last sequence being situated upstream of the sequence coding for said peptide fragment of the α1 sub-unit.

36. A process for screening molecules regulating the state of inactivation of the neuronal calcium channels involved in the release of neurotransmitters, characterized in that it comprises the following steps:
bringing together peptide fragments of the α1 sub-unit and cells expressing calcium channels for a time sufficient for the state of inactivation of the channels to be modified, then with the molecules to be tested, or bringing together cells transformed using nucleotide sequences coding for peptide fragments of the α1 sub-unit and the molecules to be tested, wherein said peptide fragments and cells are according to claim 33, and
detecting the effect of the molecules tested on the state of inactivation of the neuronal calcium channels involved in the release of neurotransmitters.

37. The screening test according to claim 22, wherein said peptide sequences are derived by mutation of one or more amino acids of the peptide fragments of the III-IV loop of the α1 sub-unit of the calcium channels, the mutation or mutations in question affecting essential amino acids within the context of the inactivation of the calcium channels at the membrane surface, to the extent that their mutation has the effect of activating or inactivating the calcium channels, for the implementation of processes for screening molecules regulating the state of inactivation of the neuronal calcium channels involved in the release of neurotransmitters.

38. The screening test according to claim 37, wherein said peptide sequences comprise one or more mutations having the effect of inactivating the calcium channels, said peptide sequences being derived by mutation of at least one of the amino acids of the peptide sequence corresponding to the sequence SEQ ID NO: 7.

39. The screening test according to claim 38, wherein said peptide sequences comprise one or more mutations having the effect of inactivating the calcium channels, said peptide sequences being derived by mutation of at least one of the amino acids situated between positions 8 and 19 of the peptide sequence corresponding to the sequence SEQ ID NO: 7, namely of at least one of the amino acids included in the sequence: DKMMEEYSLEKN.

40. A process for screening molecules regulating the state of inactivation of the neuronal calcium channels involved in the release of the neurotransmitters, characterized in that it comprises the following steps:
bringing together peptide sequences derived from the III-IV loop of the α1 sub-unit according to claim 37 and the molecules to be tested already selected for their ability to bind specifically to the non-mutated peptide sequences corresponding to said III-IV loops,
selecting said molecules binding specifically to the III-IV loops and not binding to said derived peptide sequences,
if appropriate, bringing together the molecules selected in the previous stage and cells expressing calcium channels, and observing any effect of the molecules on the regulation of the state of inactivation of the neuronal calcium channels involved in the release of neurotransmitters from said cells.

41. A peptide comprising a peptide sequence chosen from:
the sequences SEQ ID NO: 2 and SEQ ID NO: 4 in which at least one of Q in position 24, W in position 36, I in position 37, K in position 39, K in position 68, and K in position 69, is substituted by a natural or non-natural amino acid,
the sequences SEQ ID NO: 2 and SEQ ID NO: 4 in which at least one of R in position 28, R in position 63, and R in position 64, is substituted by a natural or non-natural amino acid,
the sequences SEQ ID NO: 2 and SEQ ID NO: 4 in which at least one of R in position 28, and E in position 29, is substituted by a natural or non-natural amino acid, in particular R28 is substituted by an alanine or by E, and E29 is substituted by an alanine,
the following sequence SEQ ID NO: 15: QQIERELNGYMEWISKAE.

42. A process for screening
molecules restoring the number of calcium channels to normal in cell membranes where this number has abnormally reduced, namely screening β-like molecules capable of being used in the treatment of pathologies linked to an abnormal reduction in the number of calcium channels such as epilepsy, or neuronal degeneration,
and/or molecules increasing the number of calcium channels in the cell membranes, namely screening β-like molecules capable of being used in the treatment of pathologies against which an increase in the number of calcium channels in the plasma membrane would have a beneficial effect, such as Parkinson's disease, insulin-dependent diabetes, or Lambert-Eaton myasthenic syndrome,
and/or molecules restoring the number of calcium channels to normal in the cell membranes where this number has abnormally increased, namely screening molecules capable of being used in the treatment of pathologies linked to an abnormal increase in the number of calcium channels such as cardiac hypertrophy,
and/or molecules reducing the number of calcium channels in the cell membranes, namely screening molecules capable of being used in the treatment of pathologies against which a reduction in the number of calcium channels in the plasma membrane would have a beneficial effect, such as epilepsy, hypertension, angina pectoris, or cerebral ischaemia,
and/or molecules regulating the state of inactivation of the neuronal calcium channels involved in the release of neurotransmitters, namely screening molecules capable of being used in the stimulation or inhibition of neuronal communication, in particular in the treatment of pathologies against which a regulation of the state of inactivation of the neuronal calcium channels involved in the release of neurotransmitters would have a beneficial effect, such as epilepsy, ataxia, migraine, Parkinson's disease, or cerebral ischaemia, comprising screening molecules with the screening test according to claim 22.


SEQUENCE LISTING (15 sequences)

SEQ ID NO: 1 - DNA, 384 bp, Oryctolagus cuniculus
tcaggggagtttgccaaagaaagggagcgggtggagaaccggcgcgcattcctgaagctgcggcggcagcagcagattgaacgcgagctcaacgggtacatggagtggatctcaaaagcagaagaggtgatcctcgcagaggacgagaccgacgtggagcagagacatccctttgatggagctctgcggagagccactatcaagaagagcaagacggacctgctccacccagaggaggcggaggatcagctggccgacatcgcctccgtggggtctccctttgcccgagccagcattaaaagtgccaagctggagaactcgagttttttccacaaaaaagagaggagaatgcgtttctacatccgtcgcatggtcaaaactcag

SEQ ID NO: 2 - protein, 128 aa, Oryctolagus cuniculus
SGEFAKERERVENRRAFLKLRRQQQIERELNGYMEWISKAEEVILAEDETDVEQRHPFDGALRRATIKKSKTDLLHPEEAEDQLADIASVGSPFARASIKSAKLENSSFFHKKERRMRFYIRRMVKTQ

SEQ ID NO: 3 - DNA, 384 bp, Homo sapiens
tcaggggagtttgccaaagaaagggaacgggtggagaaccggcgggcttttctgaagctgaggcggcaacaacagattgaacgtgagctcaatgggtacatggaatggatctcaaaagcagaagaggtgatcctcgccgaggatgaaactgacggggagcagaggcatccctttgatggagctctgcggagaaccaccataaagaaaagcaagacagatttgctcaaccccgaagaggctgaggatcagctggctgatatagcctctgtgggttctcccttcgcccgagccagcattaaaagtgccaagctggagaactcgaccttttttcacaaaaaggagaggaggatgcgtttctacatccgccgcatggtcaaaactcag

SEQ ID NO: 4 - protein, 128 aa, Homo sapiens
SGEFAKERERVENRRAFLKLRRQQQIERELNGYMEWISKAEEVILAEDETDGEQRHPFDGALRRTTIKKSKTDLLNPEEAEDQLADIASVGSPFARASIKSAKLENSTFFHKKERRMRFYIRRMVKTQ

SEQ ID NO: 5 - protein, 33 aa, Homo sapiens
LDFACDIYIWAPLAGTCGVLLLSLVITLYCNHR

SEQ ID NO: 6 - DNA, 165 bp, Homo sapiens
atcaccttccaggagcaaggggacaagatgatggaggaatacagcctggagaaaaatgagagggcctgcattgatttcgccatcagcgccaagccgctgacccgacacatgccgcagaacaagcagagcttccagtaccgcatgtggcagttcgtggtgtctccg

SEQ ID NO: 7 - protein, 55 aa, Homo sapiens
ITFQEQGDKMMEEYSLEKNERACIDFAISAKPLTRHMPQNKQSFQYRMWQFVVSP

SEQ ID NO: 8 - DNA, 165 bp, Oryctolagus cuniculus
atcaccttccaggagcagggcgacaagatgatggaggagtacagcttggagaaaaacgagagggcctgcatcgacttcgccatcagtgccaagccgctgaccaggcacatgccccagaacaagcagagcttccagtaccgcatgtggcagttcgtggtgtccccg

SEQ ID NO: 9 - protein, 128 aa, Oryctolagus cuniculus
  MOD_RES (24): May be Gln, Ala, or any other natural or non-natural amino acid
SGEFAKERERVENRRAFLKLRRQXQIERELNGYMEXXSXAEEVILAEDETDVEQRHPFDGALRRATIXXSKTDLLHPEEAEDQLADIASVGSPFARASIKSAKLENSSFFHKKERRMRFYIRRMVKTQ

SEQ ID NO: 10 - protein, 128 aa, Homo sapiens
  MOD_RES (24): May be Gln, Ala, or any other natural or non-natural amino acid
SGEFAKERERVENRRAFLKLRRQXQIERELNGYMEXXSXAEEVILAEDETDGEQRHPFDGALRRTTIXXSKTDLLNPEEAEDQLADIASVGSPFARASIKSAKLENSTFFHKKERRMRFYIRRMVKTQ

SEQ ID NO: 11 - protein, 128 aa, Oryctolagus cuniculus
  MOD_RES (28): May be Arg, Ala, or any natural or non-natural amino acid
SGEFAKERERVENRRAFLKLRRQQQIEXELNGYMEWISKAEEVILAEDETDVEQRHPFDGALXXATIKKSKTDLLHPEEAEDQLADIASVGSPFARASIKSAKLENSSFFHKKERRMRFYIRRMVKTQ

SEQ ID NO: 12 - protein, 128 aa, Homo sapiens
  MOD_RES (28): May be Arg, Ala, or any natural or non-natural amino acid
SGEFAKERERVENRRAFLKLRRQQQIEXELNGYMEWISKAEEVILAEDETDGEQRHPFDGALXXTTIKKSKTDLLNPEEAEDQLADIASVGSPFARASIKSAKLENSTFFHKKERRMRFYIRRMVKTQ

SEQ ID NO: 13 - protein, 128 aa, Oryctolagus cuniculus
  MOD_RES (28): May be Arg, Ala, or any other natural or non-natural amino acid
SGEFAKERERVENRRAFLKLRRQQQIEXXLNGYMEWISKAEEVILAEDETDVEQRHPFDGALRRATIKKSKTDLLHPEEAEDQLADIASVGSPFARASIKSAKLENSSFFHKKERRMRFYIRRMVKTQ

SEQ ID NO: 14 - protein, 128 aa, Homo sapiens
  MOD_RES (28): May be Arg, Ala, or any other natural or non-natural amino acid
SGEFAKERERVENRRAFLKLRRQQQIEXXLNGYMEWISKAEEVILAEDETDGEQRHPFDGALRRTTIKKSKTDLLNPEEAEDQLADIASVGSPFARASIKSAKLENSTFFHKKERRMRFYIRRMVKTQ

SEQ ID NO: 15 - protein, 18 aa, Oryctolagus cuniculus
QQIERELNGYMEWISKAE